(19) United States
(12) Patent Application Publication
(10) Pub. No.: US 2022/0338822 A1
(43) Pub. Date: Oct. 27, 2022

(54) X-RAY ASSISTIVE DEVICE FOR STANDARDIZING THE KNEE TEMPLATING PROCESS

(71) Applicant: Ohio State Innovation Foundation, Columbus, OH (US)

(72) Inventors: Mark Ruegsegger, Columbus, OH (US); Steve Starkey, Columbus, OH (US); Dylan Aery, Columbus, OH (US); Zach Reilman, Columbus, OH (US); Maddie Thew, Columbus, OH (US); Kimberly Browske, Columbus, OH (US); Kaleb Cantrell, Columbus, OH (US); Tasha Bruner, Columbus, OH (US); Ben Nowak, Columbus, OH (US); Amanda Palutsis, Columbus, OH (US); Cody Radivoyevitch, Columbus, OH (US); Zach Root, Columbus, OH (US)

(21) Appl. No.: 17/728,640

(22) Filed: Apr. 25, 2022

Related U.S. Application Data
(60) Provisional application No. 63/178,904, filed on Apr. 23, 2021.

(51) Int. Cl. A61B 6/04; A61B 6/00; A61F 2/38

(52) U.S. Cl. CPC A61B 6/0421; A61B 6/582; A61F 2/38; A61F 2002/4633

(57) ABSTRACT

Various implementations include a device for positioning a knee during an x-ray. The device includes a first portion, a second portion, and a foot plate. The first portion has a first surface, a second surface spaced apart from the first surface, a first edge extending between the first surface and the second surface, and a second edge spaced apart from the first edge. The second portion has a first surface, a second surface spaced apart from the first surface, a first edge extending between the first surface and the second surface, and a second edge spaced apart from the first edge. The first edge of the first portion is rotatably coupled to the second edge of the second portion. The foot plate is coupled to the first surface of the first portion.

### CROSS-REFERENCE TO RELATED APPLICATIONS

This application claims the benefit of U.S. Provisional Patent Application No. 63/178,904, filed Apr. 23, 2021,the content of which is incorporated herein by reference in its entirety.

### BACKGROUND

The human knee is the most complex and load-bearing joint in the body. As such, total knee arthroplasty (TKA) is the most common and complicated joint replacement surgery. With over half a million performed each year, TKA has become the most common joint replacement surgery in the United States, and that number is expected to grow 189% by 2030. With an increasing impact on the population each year, attention has been brought to optimizing this surgical process. X-rays are taken of the patient's knee prior to a TKA and placed into a templating software in order to estimate the correct sized implant to be used. Once in surgery, the true size of the knee is found and matched to the correct sized implant. The pre-operatively determined replacement size from the templating software matches the correctly sized implant only 54% of the time. This lack of efficiency results in wasted time in the operating room and creates excess transportation and storage needs.

One aspect in need of improvement is the preoperative method to template the best fitting sized implant. X-rays radiographs are the cheapest method and the most common, however, this process is inexact at many stages. As a result, preoperative measurements are not reliable and require all implant sizes to be on-hand during surgery, regardless of the templated size.

Before a knee arthroplasty, radiology technicians capture anterior-posterior and lateral radiographs of the patient's knee. The radiographs are taken to measure the knee joint and compare to-scale implant sizes to find the most appropriate fit. To establish scale in the radiographs, a metal calibration ball of known size is placed next to the knee as closely as possible. The proximity and placement of the ball relative to the joint is vital, as the ball must be in plane with and near the bony features being imaged in order to accurately scale and measure them. The error introduced through calibration ball alignment is further exaggerated by patients, who have a 100% increased risk when compared to patients with normal weight. The additional adipose tissue of obese patients that surround the knee joint increases the distance between the calibration ball and the joint and this results in a decreased accuracy of the templated implant.

Once the image is properly scaled, the surgeon analyzes the distance between the medial and lateral condyles and the distance anteriorly to posteriorly of the distal end of the femur. They then select the brand of implant and overlay each size onto the radiographed joint until an estimated correct size is determined.

The only ways to guarantee absolute accuracy in templating is to use a 3D imaging method such as a CT scan or MM, however, these techniques are expensive. When using a 2D technique such as an X-ray, the knee must be in a precise position with both the calibration ball and the X-ray generator, but this is unattainable and often a source of error. With a more standardized imaging and templating process, these errors can be eradicated to the benefit of surgeons, medical device representatives, and device companies.

The current templating process has a 54% success rate; each time the implant is incorrectly predicted, operating time is wasted opening a new implant of the correct size. Even when the implant size is correctly templated, the medical device company still needs to keep a wider range of implant sizes on hand in case the size is wrong, which creates additional storage and transportation costs. The current templating success rate is largely caused by human error intrinsic to the current X-ray process.

The goal of this project is to develop a fixture or methodology that would improve the accuracy and precision of the pre-operatively determined knee replacement size. Theoretically, this would involve constraining the knee, X-ray camera, and calibration ball (used for templating) to known and repeatable positions for the lateral and anterior-posterior X-ray scans.

In current practices, there are few standards involving the marker ball method, which is unreliable because the positioning of the element is up to the discretion of the radiology technicians. The disk and plate markers that are strapped to the body have similar limitations, but also rely on the body's morphology (i.e., a marker placed flush against the skin is not guaranteed to lie perfectly in plane with the X-ray). If the markers are not placed in-plane, the scaling is altered. For example, if the marker is placed above the plane centered at the knee joint and closer to the X-ray machine, then the joints will appear smaller than their actual size, thus incorrectly indicating an implant size that is too small for the patient's true anatomy.

Thus, there is a need for a device that assists in the standardization of anterior/posterior and lateral X-ray images taken of the knee such that the images are scalable and accurate.

### BRIEF DESCRIPTION OF THE DRAWINGS

Example features and implementations are disclosed in the accompanying drawings. However, the present disclosure is not limited to the precise arrangements and instrumentalities shown.

FIG. 1A shows a perspective view of a device for positioning a knee during an x-ray, according to one implementation.

FIG. 1B shows a perspective view of the connection of the footplate and the first portion of the device shown in FIG. 1A.

FIG. 2A shows a perspective view of the calibration ball holder of the device of FIG. 1A.

FIG. 2B shows a perspective view of a calibration ball holder, according to another implementation.

FIG. 3A shows a perspective view of the cam switch of the coupling bracket shown in FIG. 2A in the locked position.

FIG. 3B shows a perspective view of the cam switch of the coupling bracket shown in FIG. 2A in the unlocked position.

### DETAILED DESCRIPTION

The devices, systems, and methods disclosed herein provide for a new assistive device that helps place a patient's knee at the correct angle and hold the calibration ball in place during anterior/posterior and lateral X-ray images. This device stabilizes the positioning of the leg and foot of the patient during X-ray imaging and provides standardization to the templating protocol. This device improves the accuracy of templating the knee radiograph to consistently determine the appropriate tibial and femoral implant sizes prior to knee implant surgery. The device is also adjustable to fit patients of all sizes and is cost effective and easier to manufacture than possible alternatives such as a CT scan. The device is easy to use, comfortable, and scalable such that the device does not impact the current procedural preoperative timeline for total knee arthroplasty. Surgeons, medical device companies, radiology technicians, and patients can benefit from including this new device into the pre-existing X-ray imaging and templating procedures.

The devices disclosed herein do not require large changes to the imaging and templating procedure. Although it is possible to take more X-rays of the knee and marker ball or use multiple calibration balls in order to calculate 3-D aspects of the knee, such methods are unrealistic due to inefficiency. Additional and extensive training for the radiology technicians, creation of more complex software for the digital radiographs, and a significant increase in cost are some of the reasons for not designing a device that changes the normal imaging and templating procedures.

The most widely used method of imaging and templating utilizes a calibration ball that is positioned as close as possible to the condylar axis of the knee joint. The benefit of the spherical shape is that it is not subject to positioning errors as its dimensions are equal on all axes. Other markers used consist of disks or plates that can be strapped to the body, and the benefit to these is that they lie as close to the joint as the patient's anatomy allows.

The devices disclosed herein improve knee joint X-ray templating techniques through standardization and refinement of the imaging processes. This strengthens the accuracy at which tibial and femoral implant sizes are determined pre-operatively for total knee arthroplasty.

The devices disclosed herein accommodate a variety of body sizes and shapes, including weight distribution, height, width, and degrees varus-valgus of the knee joint. The devices hold the knee joint in a stable, consistent location from patient-to-patient during imaging. Specifically, the patient can be placed in a supine position and the knee held at a desirable degree of flexion that can allow for replicable trials of lateral and anterior-to-posterior X-ray images.

The devices disclosed herein are compatible with current templating and imaging software. The devices' autonomy from different templating software allows it to be useful for all brands and generations of implants. Ease of use is necessary for the technicians. The devices are easily maneuvered to achieve anterior-posterior and lateral imaging views, properly align the calibration ball for the different X-rays views, and can be easily stored. The devices are cost effective in terms of manufacturing and marketing price. Lastly, the materials chosen are radiopaque.

The devices disclosed herein increase accuracy of imaging and templating from the current mark of 54% because the devices precisely and consistently place the calibration ball in relation to the knee. For the anterior-posterior view, the calibration balls of the devices lie as close to the condylar axis as possible. For the lateral view, the calibration balls lie in the same plane as knee flexion, close to the patella. The components that hold the ball are radio-transparent so as not to interfere with the images.

The devices are easily sterilized as since they are in contact with multiple patients per day. All materials used are tolerate to common cleaning chemicals. Additionally, the device accommodates a wide range of patient sizes. The device includes adjustable length and width components to satisfy this requirement.

The devices include features that provide for ease of use by the technician that takes the same or less time to set up compared to the current calibration ball devices and are easy to store and access in the hospital setting.

The devices disclosed herein provide for the ability to be used for all body types. Additionally, the devices are not inherently connected to a specific brand or type of implant and are therefore compatible with various templating software that use a calibration ball for scaling purposes, eliminating a need for multiple devices for each brand. The devices are reusable by making them of material that can withstand heavy cleaning chemicals in between uses.

The devices disclosed herein focus on improving the placement of the calibration ball, acting as a direct improvement upon the current devices available and remaining compatible with the current calibration balls in use. This approach will minimize pushback from radiology technicians that may resist a device that requires extensive training and unfamiliar tools, as well as hospitals who want to minimize spending on new equipment. In addition, this more simplistic approach maximizes the devices' efficacy since they can be used with various joints, X-ray positions, and brands of implants. Because of this universality, the devices better meet the needs of the radiology technician for all procedures currently requiring a calibration ball and render other products unnecessary.

The devices are adjustable to accommodate patients of varying body types, including differences in patient's leg girth stemming from weight variances, height of patient's leg, and degrees of varus valgus deformities in the knee joint. The devices stabilize the patient's knee in a supine position during the radiography procedure and allow the desired degrees of flexion for correct positioning for medial lateral and anterior posterior images to be set and replicated. Thus, aiding in consistently producing accurate templating images. Additionally, the devices stabilize all attached calibration balls in the same plane as the patient's knee joint to aid in the accuracy of the templating images. The devices generate a more effective way to template the knee joint for implant sizing while not requiring a new templating software. The disclosed devices are compatible with existing templating software currently used by clinical staff. The devices increase the accuracy of implant sizing for tibial and femoral components during a total knee arthroplasty. From an economic perspective, the devices are a cost-effective addition for templating and the overall surgical procedure. From an ergonomic perspective, the devices are easily maneuverable to achieve anterior/posterior and lateral imaging views, are easily storable, and are easy to use.

The devices stabilize the knee joint so that anterior/posterior images can be taken at a 10° angle perpendicular to the long axis of the femur and ensure that in lateral images the knee is consistently in 30° flexion. After the patient's joint is stabilized, the calibration ball is set based off of the joint, and the devices prevent the calibration ball from any additional movement (+−0 mm) in any direction. The devices are adjustable to fit patients with BMI over 40 and BMI under 18. The devices also take less than two minutes to adjust and set the patient up in the correct position to stabilize the patient's joint. The devices are also foldable/collapsible and have the ability to be stored on the wall.

The devices are easily accessible, able to be regularly sterilized without degrading, are compatible with working with all body types, have dynamic parts that standardize body placement, and standardize placement for the calibration ball. The aforementioned functions help the disclosed devices maintain consistency along with not adding any difficulty for the radiology technicians. As for easily accessible, this function is most important when dealing with the radiology technicians. This function can be measured by determining the amount of time needed to use these devices, and then comparing that time to the current method used. Sterility is beneficial because of the devices' use between multiple patients during a day. This function of sterility can be quantified and verified through tests performed on the devices after use and cleaning in order to determine the amount of bacteria left on the devices. The devices being compatible with all body types is also beneficial of the large range of body types that undergo joint reconstructions. This function can be quantified by first determining both extremes of all body types, and then constructing the sizes and measurements of the devices to fit these boundaries. Next, the dynamic nature of parts of the devices to standardize body placement is important to improve consistency throughout X-rays. Making part of the devices dynamic and movable in order to fit every patient's knee at a specific angle ultimately helps solve the problem at hand. Last, the calibration ball is used in X-ray templating because it is what allows the software to accurately size the implant sizes to the X-ray. Therefore, these devices include a standardized placement of the calibration ball that is as close as possible to the knee joint while additionally being in the same plane as the joint. This standardized placement of the ball improves all consistency and accuracy throughout all X-ray templating to the knee joint.

Various implementations include a device for positioning a knee during an x-ray. The device includes a first portion, a second portion, and a foot plate. The first portion has a first surface, a second surface spaced apart from the first surface, a first edge extending between the first surface and the second surface, and a second edge spaced apart from the first edge. The second portion has a first surface, a second surface spaced apart from the first surface, a first edge extending between the first surface and the second surface, and a second edge spaced apart from the first edge. The first edge of the first portion is rotatably coupled to the second edge of the second portion. The foot plate is coupled to the first surface of the first portion.

Various other implementations include a method of performing an x-ray. The method includes providing a device for positioning a knee during an x-ray, such as the device described above. The method further includes disposing a foot of a user on the foot plate and a respective knee of the user adjacent the first edge of the first portion, disposing a calibration ball adjacent the first surface of the first portion or the first surface of the second portion, and producing an x-ray image that includes the knee of the user and the calibration ball.

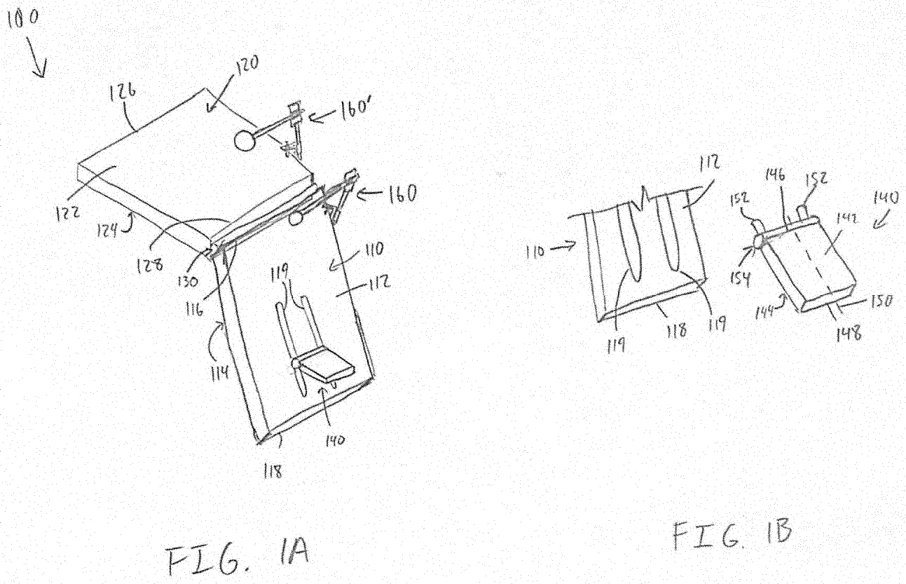

FIG. 1 shows a device 100 for positioning a knee during an x-ray, according to aspects of various implementations. The device 100 includes a first portion 110, a second portion 120, a foot plate 140, a first calibration ball holder 160, and a second calibration ball holder 160′.

The first portion 110 has a first surface 112, a second surface 114 spaced apart from the first surface 112, a first edge 116 extending between the first surface 112 and the second surface 114, and a second edge 118 spaced apart from the first edge 116.

The second portion 120 has a first surface 122, a second surface 124 spaced apart from the first surface 122, a first edge 126 extending between the first surface 122 and the second surface 124, and a second edge 128 spaced apart from the first edge 126.

The second edge 128 of the second portion 120 includes a hinge 130 that is rotatably coupled to the first edge 116 of the first portion 110. The first surface 112 of the first portion 110 and the first surface 122 of the second portion 120 form a knee angle, wherein the first portion 110 is rotatable relative to the second portion 120 such that the knee angle can be from 0 degrees to 20 degrees.

The foot plate 140 includes a first foot surface 142, a second foot surface 144 spaced apart from the first foot surface 142, a heel portion 146 extending between the first foot surface 142 and the second foot surface 144, a toe portion 148 opposite and spaced apart from the heel portion 146, and a foot plate axis 150 extending between the heel portion 146 and the toe portion 148. The foot plate 140 is coupled to the first surface 112 of the first portion 110 such that the heel portion 146 is closer than the toe portion 148 to the first surface 112 of the first portion 110 and the first foot surface 142 is closer than the second foot surface 144 to the first edge 116 of the first portion 110.

The foot plate 140 is movable between a first position and a second position. In the first position, the foot plate 140 is closer to the first edge 116 of the first portion 110 than when it is in the second position. To accomplish this motion, the first surface 112 of the first portion 110 defines two slots 119, and the foot plate 140 includes two protrusions 152 that are each disposed within a separate one of the slots 119.

The angle of the foot plate 140 is further adjustable to allow for different angles of the patient's foot relative to the first surface 112 of the first portion 110. The foot plate axis 150 forms a foot angle with the first surface 112 of the first plate 110, and the foot plate 140 includes a hinge 154. The foot plate 140 is rotatably coupled to the first surface 112 of the first portion 110 by the hinge 154 such that the foot angle can be from 90 degrees to 45 degrees.

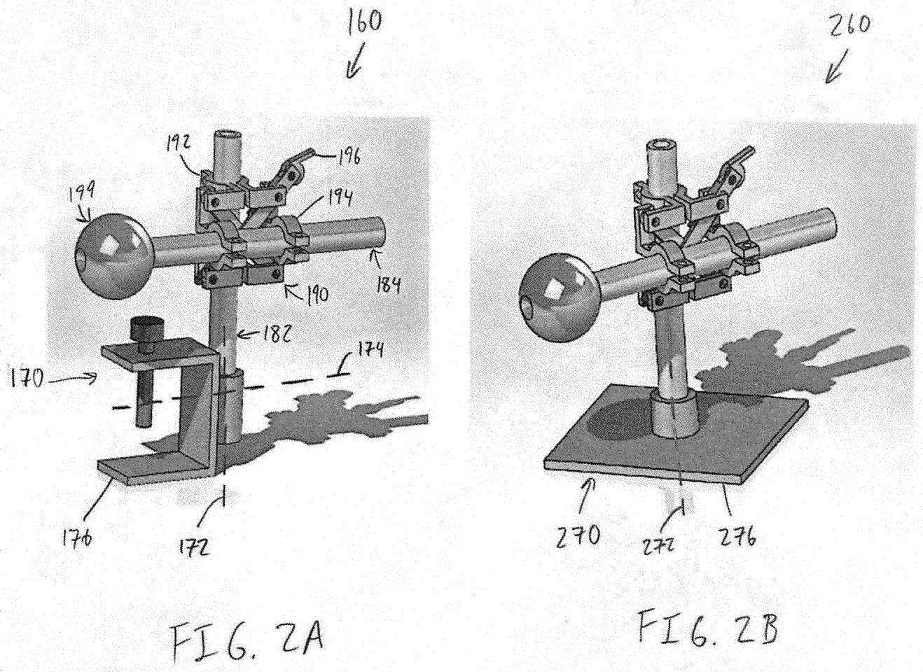

The first calibration ball holder 160 is couplable to the first surface 112 of the first portion 110, and the second calibration ball holder 160′ is coupled to the first surface 122 of the second portion 120. As shown in FIGS. 2A and 2B, each calibration ball holder 160 includes a base 170, a first support 182, a second support 184, a coupling bracket 190, and a radiopaque calibration ball 199.

The base 170 is configured to be mounted to a mounting surface, such as the first surface 112 of the first portion 110 or the first surface 122 of the second portion 120. The base 170 defines a z-axis 172 extending away from and normal to the mounting surface 112, 122 when the base 170 is mounted to the mounting surface 112, 122. The base 170 further defines an x-axis 174 extending perpendicular to the z-axis 172.

The base 170 of the calibration ball holder 160 shown in FIG. 2A includes a mounting clamp 176 configured to be couplable to a mounting surface 112, 122. The base 170 is configured such that the z-axis 172 extends away from the mounting surface 112, 122 when the base 170 is mounted to the mounting surface 112, 122.

The base 270 of the calibration ball holder 260 shown in FIG. 2B includes a mounting stand 276 configured to be supported to a mounting surface 112, 122. The base 270 is configured such that the z-axis 272 extends away from the mounting surface 112, 122 when the base 270 is mounted to the mounting surface 112, 122.

The first support 182 of the calibration ball holder 160 shown in FIG. 2A extends longitudinally parallel to the z-axis 172 and is coupled to the base 170. The second support 184 extends longitudinally parallel to the x-axis 174 and is coupled to the first support 182 by the coupling bracket 190. The calibration ball 199 is coupled to an end of the second support 184.

The coupling bracket 190 includes a first clamp 192, a second clamp 194, and a cam switch 196. The first clamp 192 is configured to releasably couple the coupling bracket 190 to the first support 182, and the second clamp 194 is configured to releasably couple the coupling bracket 190 to the second support 184.

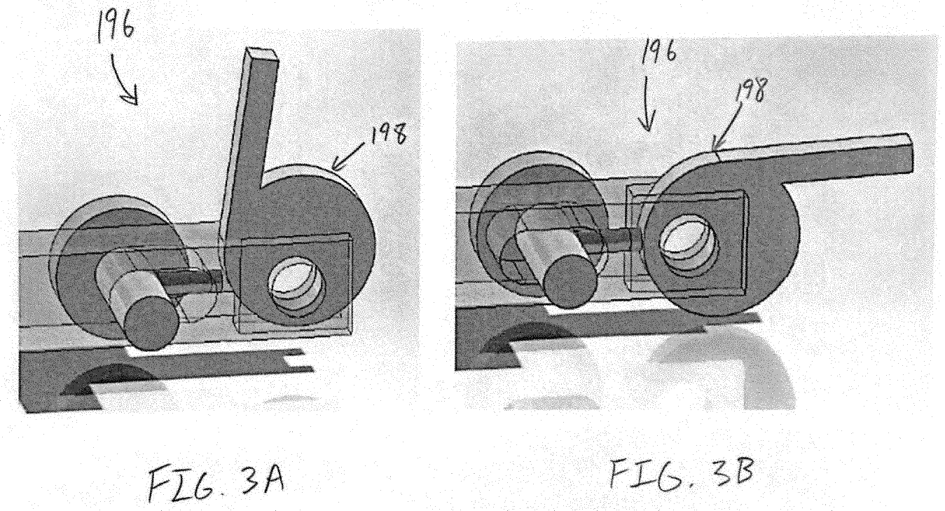

The cam switch 196 of the coupling bracket 160 is shown in FIGS. 3A and 3B. The cam switch 196 is rotatable from a locked position (shown in FIG. 3A) to an unlocked position (shown in FIG. 3B). When the cam switch 196 is in the locked position, a cam 198 of the cam switch 196 causes the first clamp 192 and the second clamp 194 to be rigidly coupled to the first support 182 and the second support 184, respectively. When the cam switch 196 is in the unlocked position, the cam 198 of the cam switch 196 causes the first clamp 192 and the second clamp 194 to be slidingly and rotatably coupled to the first support 182 and the second support 184, respectively.

When the cam switch 196 is in the unlocked position, the calibration ball 199 is able to be slidingly moved parallel to the z-axis 172 by moving the coupling bracket 190, second support 184, and calibration ball 199 relative to the first support 182. The calibration ball 199 can also be slidingly moved parallel to the x-axis 174 by moving the second support 184 and calibration ball 199 relative to the coupling bracket 190 and the first support 182. Furthermore, the calibration ball 199 can be rotated in a circumferential direction about the z-axis 172 by rotating the coupling bracket 190, second support 184, and calibration ball 199 relative to the first support 182. Thus, the calibration ball 199 can be moved to any position needed by the user by moving the cam switch 196 from the locked position to the unlocked position. Once the calibration ball 199 is in the desired position, the position can be selectively and rigidly held in the position by moving the cam switch 196 to the locked position.

In some implementations, the calibration ball is coupled to the second support by a flexible claw fixture. The flexible claw fixture allows for calibration balls of different sizes (e.g., 25 mm, 30 mm, 1 inch) to be used with the same device.

However, in other implementations, one or both of the calibration ball holders can include a suction cup to allow the calibration ball holder to be movably coupled to one of the first surfaces of the first portion or the second portion by suction.

In use, the foot of a user is disposed on the first foot surface 142 of the foot plate 140 such that the respective knee of the user is disposed adjacent the first edge 116 of the first portion 110 at the hinge 130. The foot plate 140 can be adjusted to account for the size and length of the lower leg of the user to ensure that the knee is disposed adjacent the hinge point between the first portion 110 and the second portion 120.

Next, a first calibration ball holder 160 is moved and coupled to the first surface 112 of the first portion 110 adjacent the knee of the user. If a second calibration ball is needed or desired in the x-ray, then a second calibration ball holder 160′ can be moved and coupled to the first surface 122 of the second portion 120 to a desired position.

If angulation of the foot is desired, then the foot angle of the foot plate 140 can be adjusted.

After the leg and foot of the user are positioned and the calibration ball holders 160, 160′ have been moved to their desired locations, an x-ray image of the user's knee and the one or more calibration balls 199 can be produced. In some implementations, the x-ray image may be produced as an anterior x-ray image and/or a lateral x-ray image.

In some implementations, the device includes features to add comfortability, the ability to capture images while the patient is laying down or standing, and the ability adjust the leg to correct standard angles. To make the device comfortable for the patient, memory foam or silicone is coupled to the interior of the design. This level of comfort can be tested through many trials in the development stage of creating the device. Another function that improves the look and performance of the device is the device being adjustable from the wall or moved on the X-ray table. This feature allows the device to have a nice appearance in the radiology room and helps the accessibility for radiology technicians.

In some implementations, the device captures X-ray images with the patient laying on the table, squatting on a stool, or standing up. The device standardizes the image taking process, so the change in position of the person does not affect the outcome. This feature also makes image capturing more straight forward for the radiology tech if the patient has a disability or is simply unable to position themselves in one way.

In some implementations, the device includes a dial to indicate the exact angle of the knee joint. The dial helps with standardization and consistency while using this device, and it allows physicians to record the angle that the X-ray was performed at for future analysis.

The disclosed device has the ability to be used for all body types. Because the device is one-size-fits-all, only a single device is needed in the X-ray imaging room. Additionally, the device includes materials that are able to withstand heavy cleaning chemicals in between uses. This ensures that the lifecycle of the device will be long and that the device will not need to be replaced often. The device also includes materials that will last for many years to decrease waste.

Another way that the device decreases waste is through accurate templating after the X-rays are taken with the device because the device helps to ensure proper sizing of a knee implant. Thus, there is less need for excess materials and implant sizes brought into the operating room during surgery.

In some implementations, the device can detach from a wall and then placed on a patient in some type of restraint. The goal of the device is to minimize discrepancies caused by movement or fluctuating knee angles such as inverted, or “knock knees,” and bowing knees. In some cases, the preferred angle of the knee for the X-ray template is about 20 degrees. If the knee bows one way or the other, it can lead to size discrepancies when templating depending on the plane, so the devices disclosed herein are configured to standardize these angles. Furthermore, calibration balls typically come in one size (30 mm) and are often held in position via an adjustable arm. In some implementations, the device includes a brace with a calibration ball attached next to the knee and an adjustable arm that can attach to the device that slides either in front of the knee or to the side of the knee. In some implementations, multiple smaller calibration balls are used to normalize the calibration ball size from the various planes. In some implementations, the device holds the knee in a sitting position.

In other implementations, the device includes a laser alignment feature, a locking knee-brace, and/or a calibration ball attached to a stretchy looped material.

In one implementation, the device includes feet and leg directors that can be used while a patient is in supine position. These directors allow rotational adjustments to be made in order to place feet and legs in the correct positions and angles.

The foot and leg directors can each include a long leg directing component attached to a foot holder. This foothold positions the patient's foot at the desired angle for the intended X-ray view. The leg portion pivots to be set at the desired angle as well. Additionally, the foot and leg directors include foam padding to increase patient comfort. This implementation also includes incorporating the calibration ball into the leg guide by attaching the calibration ball to a built-in rod that could slide up and down the device as well as in and out to be placed at the closest position and plane of the knee as possible.

In another implementation, the device can attach to the leg and hold the calibration ball. This implementation of the device fits over the knee and is adjustable to allow the ball to be moved to the correct medial/lateral, posterior/anterior, and superior/inferior positions.

In yet another implementation, the device can include multiple calibration balls to aid in improving scaling and placement in the correct plane.

Furthermore, the device can include adjusting features that allow the device to extend and retract to fit different heights of patients. The disclosed device is also able to incorporate multiple calibration balls. The ability of the device to fit a large variety of heights is important because of the large variety and population of patients that undergo orthopedic joint replacement surgeries per year. This feature allows the device to be adaptable to many different patients. Additionally, the device's ability to incorporate and use multiple different calibration balls is important for the performance of the device. A calibration ball is important in X-ray templating because the calibration balls allow the software to size the implants appropriately for templating. With the ability of the device to incorporate multiple calibration balls, the device is compatible with the templating software. Additionally, with multiple calibration balls, the device and templating process should be more accurate. The calibration balls are able to be incorporated into the main device via a small piece that holds the calibration balls in specific locations. This calibration ball piece is attached to the device via a suction cup like feature. This feature allows the separate piece to be placed in the appropriate location for each patient. Additionally, FIG. [6] shows how the device fits to a patient's leg for X-rays. All of these features that are selected and incorporated into the device help improve the consistency of X-ray images, performance of the device, and the capability of the device.

A number of example implementations are provided herein. However, it is understood that various modifications can be made without departing from the spirit and scope of the disclosure herein. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various implementations, the terms “consisting essentially of” and “consisting of” can be used in place of “comprising” and “including” to provide for more specific implementations and are also disclosed.

Disclosed are materials, systems, devices, methods, compositions, and components that can be used for, can be used in conjunction with, can be used in preparation for, or are products of the disclosed methods, systems, and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a device is disclosed and discussed each and every combination and permutation of the device are disclosed herein, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

1. A device for positioning a knee during an x-ray, the device comprising:
a first portion having a first surface, a second surface spaced apart from the first surface, a first edge extending between the first surface and the second surface, and a second edge spaced apart from the first edge;
a second portion having a first surface, a second surface spaced apart from the first surface, a first edge extending between the first surface and the second surface, and a second edge spaced apart from the first edge, wherein the first edge of the first portion is rotatably coupled to the second edge of the second portion; and
a foot plate coupled to the first surface of the first portion.

2. The device of claim 1, further comprising a calibration ball holder couplable to the first surface of the first portion or the first surface of the second portion.

3. The device of claim 2, wherein the calibration ball holder is movably couplable to the first surface of the first portion or the first surface of the second portion.

4. The device of claim 2, wherein the calibration ball holder is couplable to the first surface of the first portion or the first surface of the second portion by suction.

5. The device of claim 2, wherein the calibration ball holder is a first calibration ball holder, the device further comprising a second calibration ball holder couplable to the first surface of the first portion or the first surface of the second portion.

6. The device of claim 1, wherein the foot plate is movable between a first position and a second position, wherein the first position is closer than the second position to the first edge of the first portion.

7. The device of claim 1, wherein the foot plate includes a first foot surface, a second foot surface spaced apart from the first foot surface, a heel portion extending between the first foot surface and the second foot surface, a toe portion opposite and spaced apart from the heel portion, and a foot plate axis extending between the heel portion and the toe portion, wherein the heel portion is closer than the toe portion to the first surface of the first portion, and the first foot surface is closer than the second foot surface to the first edge of the first portion.

8. The device of claim 7, wherein the foot plate axis forms a foot angle with the first surface of the first plate, wherein the foot plate is rotatably coupled to the first surface of the first portion such that the foot angle can be from 90 degrees to 45 degrees.

9. The device of claim 1, wherein the first surface of the first portion and the first surface of the second portion form a knee angle, wherein the first portion is rotatable relative to the second portion such that the knee angle can be from 0 degrees to 20 degrees.

10. The device of claim 2, wherein the calibration ball holder comprises:
a base configured to be mounted to one of the first surface of the first portion or the first surface of the second portion, wherein the base defines a z-axis extending away from the one of the first surface of the first portion or the first surface of the second portion when the base is mounted to the one of the first surface of the first portion or the first surface of the second portion;
a support extending longitudinally parallel to the z-axis;
a calibration ball slidingly coupled to the support such that the calibration ball can be selectively moved in a direction parallel to the z-axis, wherein the calibration ball is radiopaque.

11. The device of claim 10, wherein the z-axis is normal to the one of the first surface of the first portion or the first surface of the second portion when the base is mounted to the one of the first surface of the first portion or the first surface of the second portion.

12. The device of claim 10, wherein the base further defines an x-axis perpendicular to the z-axis, wherein the support is a first support, the device further comprising a second support extending longitudinally parallel to the x-axis, wherein the calibration ball is slidingly coupled to the first support by being coupled to the second support.

13. The device of claim 12, wherein the second support is slidingly coupled to the first support such that the calibration ball can be selectively moved in a direction parallel to the x-axis.

14. The device of claim 13, wherein the second support is rotatably coupled to the first support such that the calibration ball can be selectively rotated in a circumferential direction about the z-axis.

15. The device of claim 13, wherein the second support is coupled to the first support by a coupling bracket, the coupling bracket including a first clamp and a second clamp, the first clamp being configured to releasably couple the coupling bracket to the first support, and the second clamp being configured to releasably couple the coupling bracket to the second support.

16. The device of claim 15, wherein the coupling bracket further includes a cam switch, wherein the cam switch is rotatable from a locked position to an unlocked position, wherein, when the cam switch is in the locked position, a cam of the cam switch causes the first clamp, the second clamp, or both to be rigidly coupled to the first support, the second support, or both, respectively, and wherein, when the cam switch is in the unlocked position, the cam of the cam switch causes the first clamp, the second clamp, or both to be slidingly coupled to the first support, the second support, or both, respectively.

17. The device of claim 16, wherein, when the cam switch is in the locked position, the cam of the cam switch causes the first clamp and the second clamp to be rigidly coupled to the first support and the second support, respectively, and wherein, when the cam switch is in the unlocked position, the cam of the cam switch causes the first clamp and the second clamp to be slidingly coupled to the first support and the second support, respectively.

18. The device of claim 10, wherein the base comprises a mounting clamp configured to be couplable to the one of the first surface of the first portion or the first surface of the second portion such that the z-axis extends away from the one of the first surface of the first portion or the first surface of the second portion when the base is mounted to the one of the first surface of the first portion or the first surface of the second portion.

19. The device of claim 10, wherein the base comprises a mounting stand configured to be supported by the one of the first surface of the first portion or the first surface of the second portion such that the z-axis extends away from the one of the first surface of the first portion or the first surface of the second portion when the base is mounted to the one of the first surface of the first portion or the first surface of the second portion.

20.-49. (canceled)
